(19) United States
(12) Patent Application Publication
(10) Pub. No.: US 2025/0189418 A1
(43) Pub. Date: Jun. 12, 2025

(54) METHOD FOR CONCENTRATING AT LEAST ONE TARGET SUBSTANCE IN A VOLUME OF LIQUID

(71) Applicant: IST Innuscreen GmbH, Berlin (DE)

(72) Inventors: Timo Hillebrand, Hönow (DE); Elmara Graser, Berlin (DE); Wiebke Jacobi, Falkensee (DE)

(21) Appl. No.: 18/706,409

(22) PCT Filed: Nov. 17, 2022
(86) PCT No.: PCT/EP2022/082182

§ 371 Date: May 1, 2024

(30) Foreign Application Priority Data
Nov. 22, 2021 (DE) 10 2021 130 537.3
Nov. 24, 2021 (DE) 10 2021 130 831.3
Dec. 7, 2021 (DE) 10 2021 132 214.6
Feb. 15, 2022 (DE) 10 2022 103 554.9

(51) Int. Cl. G01N 1/40; B01J 20/26; B01J 20/28; G01N 33/50

(52) U.S. Cl. CPC G01N 1/405; B01J 20/26; B01J 20/28004; B01J 20/28016; G01N 33/50

(57) ABSTRACT

A method for producing a sample that contains a target substance from a first volume of liquid that contains the target substance comprises adding a superabsorber to the first volume of liquid, incubating the first mixture of the superabsorber and the first volume of liquid, and removing a sample of the first mixture present after incubation. An additional method for producing a sample that contains a target substance from a first volume of liquid of a sample liquid that contains the target substance comprises adding a superabsorber to the first volume of liquid, incubating the first mixture of the superabsorber and the first volume of liquid, adding a second volume of an aqueous solution to the remaining superabsorber, incubating the second mixture of the superabsorber and the second volume of liquid, and removing a sample of the second mixture present after incubation.

The invention relates to a method for concentrating at least one target substance in a volume of liquid and for generating a sample containing at least one target substance.

The detection of target substances, in particular of biological target substances, plays an important role in liquid analysis. Target substances to be detected or monitored can be biomolecules, such as eukaryotic cells, prokaryotic cells, subcellular vesicles, bacteriophages, viruses, toxins, antibodies, or also nucleic acids or proteins. Analysis methods are used for the qualitative or quantitative determination of target substances in samples taken from a liquid to be examined. For this purpose, laboratory methods that can be carried out manually or in an automated manner or at least partially in an automated manner, or analytical instruments operating in a completely automated manner, are available. The analytical instruments operating in an automated manner also include online analytical instruments, which remove samples continuously or discontinuously from the liquid to be monitored and carry out a qualitative or quantitative determination of the target substance.

At low concentrations of the target substance in the liquid to be analyzed, the problem arises of providing a sufficient amount of the target substance for the subsequent analysis during sampling. In some applications, the target substance is present in an untreated liquid sample at a concentration which is too low for the subsequent processing or analysis. This results in the need for concentration of the target substance in the sample. An example of this is the detection of biomolecules in water or wastewater, for example the detection of SARS-COV-2 in water by means of molecular genetic techniques, such as PCR or real-time PCR. The concentrations of the virus particles to be detected or of viral fragments in the wastewater are often too low for them to be detected by means of the methods known to a person skilled in the art. If a volume of 200 μl is sufficient when examining blood samples for the presence of a virus infection, the required sample volume for the examination of water/wastewater is significantly greater. The literature describes starting amounts of up to several liters.

The concentration of target molecules in a volume of liquid plays an important role not only for the preparation of samples for molecular genetic analysis techniques, but equally also for all immunological technologies and spectroscopic technologies, such as molecular spectroscopy or mass spectroscopy, for example.

The concentration of target substances, in particular biomolecules, in a volume of liquid also plays a role in the generation of crude products or products comprising the target substances or biomolecules, which serve for further use for research purposes, for industrial purposes or for therapeutic purposes.

In the prior art, various techniques are known for the enrichment of viruses or subcellular particles from a biological sample, for example ultracentrifugation techniques or the technique of ultrafiltration. These methods are time-consuming and relatively expensive. Alternative processes consist in the precipitation of virus particles by means of polyethylene glycol/sodium chloride and subsequent centrifugation (Yamamoto et al., Virology 40 (1970) 734; Mordi et al., J. Clin. Microbiol. 36 (1998) 1543-1538). Various mixtures of PEG and sodium chloride are used, and these reagents are mixed with the biological sample. Subsequently, the batch is incubated for a longer period of time at cold and subsequently the virus (protein) NaCl/PEG precipitates are obtained by centrifugation. These methods are also complicated and require a lot of time. Furthermore, the further processing of the precipitates for the isolation of the viral nucleic acids is problematic. The precipitates can often be brought into solution again only with great difficulty. This considerably influences the efficiency and the quality of the nucleic acid isolation.

From US 2015/0224502 A1, a sample collection device is known for flow-through sampling in waters. Here, for concentration of target substances present in waters at low concentration, the approach is taken to retain the target substances in a filter or adsorption medium and to release the substances adsorbed at the filter by elution or as a lysate for subsequent analysis and to provide them to an analysis module. This method is also complex in terms of apparatus and cannot be used universally.

The object of the invention is to provide a simple, fast and universally usable method for producing a sample containing at least one target substance from a volume of a sample liquid containing the at least one target substance for the subsequent analysis or as a crude product or product for subsequent use for research purposes, for therapeutic purposes or for industrial purposes, for example for product production.

This object is achieved in a surprisingly simple and universally applicable manner by means of the method defined in claim 1. Likewise, the object is achieved by means of the method defined in claim 2. The object is also achieved by the use specified in claim 18 and the kit specified in claim 20. Advantageous embodiments are listed in the dependent claims.

The method according to the invention for producing a sample containing at least one target substance from a first volume of liquid of a sample liquid containing the at least one target substance by concentration of the at least one target substance in the first volume of liquid comprises:
adding a superabsorber to the first volume of liquid or adding the first volume of liquid to the superabsorber,
incubating the first mixture formed from the superabsorber and the volume of liquid, and
removing a sample of the liquid fraction of the first mixture present after incubation.

A further method according to the invention for producing a sample containing at least one target substance from a first volume of liquid of a sample liquid containing at least one target substance comprises:
adding a superabsorber to the first volume of liquid or adding the first volume of liquid to the superabsorber,
incubating the first mixture formed from the superabsorber and the first volume of liquid, in particular until substantially complete disappearance of the liquid fraction of the first mixture,
then adding a second volume of liquid of an aqueous solution to the remaining superabsorber or adding the remaining superabsorber to the second volume of liquid and thus producing a second mixture of the superabsorber and the second volume of liquid, and
removing a sample of the liquid fraction of the second mixture.

The second mixture formed from the superabsorber and the second volume of liquid can be incubated before the sample of the liquid fraction of the second mixture is removed, for example in order to set a specific concentration range of the target substance or a certain volume of the liquid fraction.

The aqueous liquid added in this method after incubation of the first mixture can, for example, comprise a lysis buffer.

The incubation of the first mixture formed from the superabsorber and the first volume of liquid can be carried out until there is a great reduction in the liquid fraction of the first mixture. The liquid fraction can also completely disappear or at least be reduced so far that no more liquid can be absorbed from the mixture by means of a pipette or a dabber.

In both methods according to the invention, a sample containing at least one target substance is produced and can subsequently be analyzed and/or can be further used as a crude product or product in a process or method. In this case and in the following, the term “sample” is therefore used to denote a substance volume, for example a liquid substance volume, which contains the target substance in a concentration that can be influenced or adjusted during the production of the sample according to the invention. The sample can thus not only be available for a subsequent analysis, but it can also be used as a crude product or product for further research purposes or also for, for example industrial, product production or for therapeutic purposes. Such samples serving as a crude product or product can comprise, for example, concentrated viruses, nucleic acids, antibodies or proteins.

Plastics which are capable of absorbing a multiple of their own weight in polar liquids are referred to as superabsorbers (also: superabsorbent polymers, SAP). Polar solvents such as, for example, water or aqueous solutions can be considered as liquids suitable for absorption by the superabsorber. When the liquid is absorbed, the superabsorber swells and forms a hydrogel. Hydrogels can form all cross-linked polymers which are polar, for example polyacrylamide, polyvinylpyrrolidone, amylopectin, gelatin, cellulose. However, plastics, in particular the plastics mentioned here and below, are preferred compared to biological polymers for the present invention.

Suitable for the invention is, for example, a copolymer of acrylic acid (propene acid, H₂C═CH—COOH) or sodium acrylate (sodium salt of acrylic acid, H₂C═CH—COONa) on the one hand and acrylamide on the other hand, wherein the ratio of the two monomers to one another can vary. Other polyacrylates or other polymers or copolymers based on acrylic acid or acrylate as monomer are also possible. In addition, in the preparation of the copolymer, a so-called core cross-linker (CXL) of the monomer solution can be added, which connects (cross-links) the formed long-chain polymer molecules in places to one another by chemical bridges. These bridges render the polymer insoluble in water. This so-called base polymer is optionally subjected to a so-called surface post-cross-linking (surface cross-linking, SXL). A further chemical is applied here to the surface of the particles which, by heating, links a second network only to the outer layer of the grain. This shell supports the swollen gel in order to also hold together under external loading (movement, pressure).

Superabsorbers in the form of granules are used, for example, conventionally in baby diapers, monthly hygiene products, in incontinence supply and in dressing material. The use in cable sheathing for deep-sea lines is also known. Furthermore, superabsorbers are used as gel-forming extinguishing agents in fire control, as mechanical stabilizers for cut flowers in a vase or as additive for plant earth for permanent water storage. Because of the better environmental compatibility, the use of potassium-neutralized acrylic acid is used here. In the form of spherical particles, the use of superabsorbers as a toy under designations such as “water pearls”, “aqua beads” or “water beads” is known. These are superabsorbers which are commercially available in the form of beads of variable sizes from submillimeters to centimeters.

As will be described in more detail further below, it has surprisingly been found that, although a high proportion of the liquid is absorbed by the superabsorber during the incubation of a mixture of such a superabsorber and a sample liquid containing at least one, in particular biological, target substance, the target substance remains in the liquid fraction not absorbed by the superabsorber, or, with substantially complete disappearance of the liquid fraction, remains on the surface of the superabsorber. Since the target substances, in particular biological target substances, are therefore not absorbed by the superabsorber, the concentration of the at least one target substance is substantially proportional to the concentration of the target substance in the original sample liquid in a liquid fraction of the first mixture remaining after incubation or the concentration of the target substance in a solution which is formed by adding an aqueous solution to the superabsorber remaining after incubation of the first mixture. This allows a quantitative determination of the concentration of the target substance in the sample liquid on the basis of the sample produced by the method according to the invention. This effect can also be used for a targeted setting of a desired concentration of the target substance in a sample which serves as a crude product or product for further research purposes, therapy purposes, or product preparation.

In the method according to the invention, the first volume of liquid can contain a polar liquid, in particular as a main constituent. In an advantageous embodiment of the method according to the invention, the first volume of liquid can contain a polar solvent, in particular as a main constituent. For example, the first volume of liquid can consist of a polar solvent to a mass fraction of at least 50%. The polar liquid or the polar solvent can be water, for example.

The target substance can be a biomolecule. It can be selected from the following substances: eukaryotic cells, constituents of eukaryotic cells, prokaryotic cells, components of prokaryotic cells, subcellular vesicles, bacteriophages, viruses, or virus components, toxins, antibodies, nucleic acids, and proteins.

As mentioned, in an advantageous embodiment, the superabsorber can be a plastic or comprise a plastic which absorbs a proportion of the volume of liquid, for example a polar solvent contained in the volume of liquid, such as water, to form a gel or hydrogel. Advantageously, the plastic is selected such that it substantially does not take up any biomolecules or the substances specified further above, such as eukaryotic cells, constituents of eukaryotic cells, prokaryotic cells, components of prokaryotic cells, subcellular vesicles, bacteriophages, viruses or virus components, toxins, antibodies, nucleic acids, and proteins. The superabsorber is therefore intended to absorb the target substance, e.g., biomolecules, not at all or only to a negligible proportion for the purpose of the enrichment of the target substance in the sample to be produced. This is the case, for example, for the above-mentioned superabsorbers from the stated polymer or copolymer materials, for example in the case of the commercially available water pearls, water beads, etc.

The superabsorber can be used in the form of particles, for example as a powder, as granules or in the form of geometric bodies, in particular beads (spherical particles). It can thus be added to the volume of liquid in the form of such particles, or the volume of liquid can be added to the superabsorber in this form. The particles or beads can have a diameter between 100 to 5000 μm.

Advantageously, the superabsorber is constituted by commercially available superabsorber beads, for example superabsorber beads by the name of “aquabeads”, “water beads”, “water pearls”, “aquapearls”, “hydrobeads”, “gel beads”

In an advantageous embodiment of the method, the volume of the liquid fraction of the above-mentioned first and/or second mixture remaining after the step of incubation, and thus the concentration of the target substance in the remaining liquid fraction, by the duration of incubation, can be controlled by the size and number of the superabsorber particles or superabsorber balls formed of the superabsorber added to the volume of liquid, and/or by the temperature prevailing during incubation.

The invention also comprises a use of a superabsorber for concentrating a target substance, in particular a biomolecule, in a volume of liquid. The volume of liquid can contain a polar liquid, in particular water, wherein the superabsorber is configured to absorb the polar liquid, e.g., water, or at least a portion of the polar liquid, with formation of a hydrogel. The superabsorber can be formed from the materials described above and can be used in the above-described embodiments as granules or, particularly preferably in the form of beads, in particular commercially available water pearls, water beads, or aqua beads. The use may comprise adding the superabsorber to the volume of liquid or adding the volume of liquid to the volume of liquid and incubating the mixture thus formed, as explained further above with reference to the described methods according to the invention. The use can also be the control of a temperature and/or an incubation time in order to selectively adjust a concentration or a concentration range of the target substance in the liquid fraction of the mixture. Very generally, the invention comprises a method for concentrating a superabsorber for concentrating a target substance, in particular a biomolecule, in a volume of liquid by adding a superabsorber to the volume of liquid or by adding the volume of liquid to the superabsorber and incubating it. A sample removed after incubation from the remaining liquid fraction of the mixture of the liquid and the superabsorber can serve for further analysis or for further use, for example in a production process or for therapeutic purposes or for research purposes. The volume of the sample removed can substantially correspond to the volume of the remaining liquid fraction or be significantly lower than the volume of the remaining liquid fraction.

The invention also comprises a kit for carrying out the methods and/or method variants described above. The kit can comprise the superabsorbers and further substances and/or means for carrying out the method. The kit can, for example, comprise at least one container with a superabsorber, into which an initial volume of liquid can be added for concentration.

The sample obtained according to the described methods or according to the described use can be supplied manually or in an automated manner to a laboratory instrument for further treatment or analysis. The further analysis can be carried out by means of molecular analysis techniques, immunological technologies and spectroscopic technologies, for example molecular spectroscopy or mass spectroscopy, for example by means of optical or electrochemical sensors, by cultivation, sequencing or flow cytometry.

The invention accordingly also includes a method for the qualitative or quantitative determination of at least one target substance in a sample liquid, comprising:
producing a sample containing the at least one target substance from a first volume of liquid of a sample liquid containing the at least one target substance according to one of the above-described methods according to the invention in one of the above-described embodiments, and
qualitatively or quantitatively detecting the at least one target substance in the sample by means of at least one of the following methods: nucleic acid-based detection methods, sequencing, immunological detection methods, microbiological analyses, microscopic methods, mass spectrometry, sensor-based evidence and flow cytometric techniques.

As in the methods and method embodiments described above, the target substance can be one of the biological substances mentioned further above.

Nucleic-acid-based detection methods can comprise, for example, PCR, real-time PCR-based methods, digital PCR-based methods or sequencing. Immunological detection methods can be immunological assays, such as ELISA or lateral flow tests. Microbiological analyses can comprise the cultivation of living cells in the sample. Sensor-based evidence can comprise detection methods by means of optical, spectroscopic or electrochemical sensors.

In one possible embodiment of the method or the use, the produced sample or the further-treated sample can be added to a cartridge, in particular a microfluidic cartridge, of an automatic analysis device for the automated performance of a detection of the target substance by means of molecular genetic techniques, such as PCR or real-time PCR. This can take place manually or in an automated manner.

The invention also includes a kit for carrying out the described method for qualitative or quantitative determination of at least one target substance in the sample liquid. This kit can contain at least the superabsorber in a dosage form suitable for the method and optionally further chemicals, such as buffer solutions or lysis buffers.

The invention is explained in more detail below with reference to the figures and some exemplary embodiments. These examples do not represent any limitation of the agents and methods according to the invention.

In the figures:

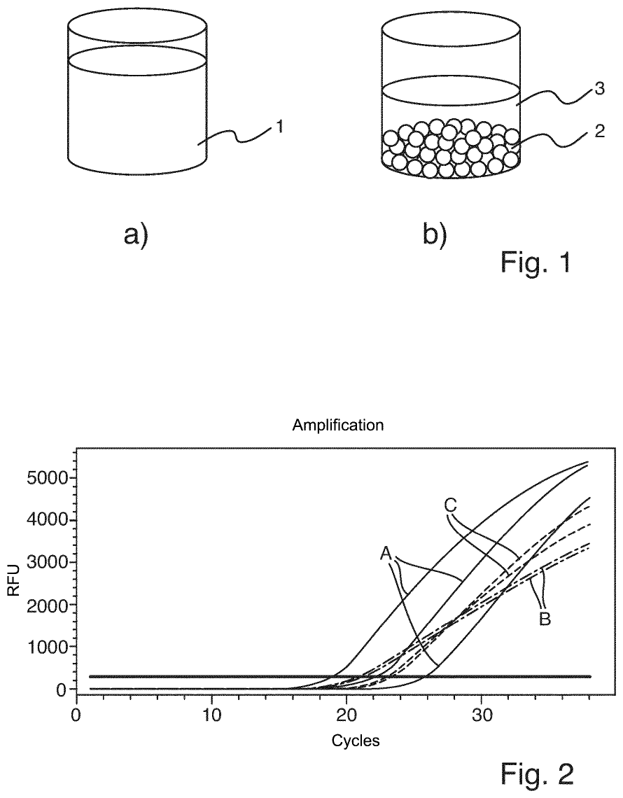

FIG. 1 shows a schematic representation of a liquid before (a) and after addition of a superabsorber in the form of beads and incubation (b);

FIG. 2 shows amplification curves of different samples from surface water without concentration and after concentration by means of the method according to the invention;

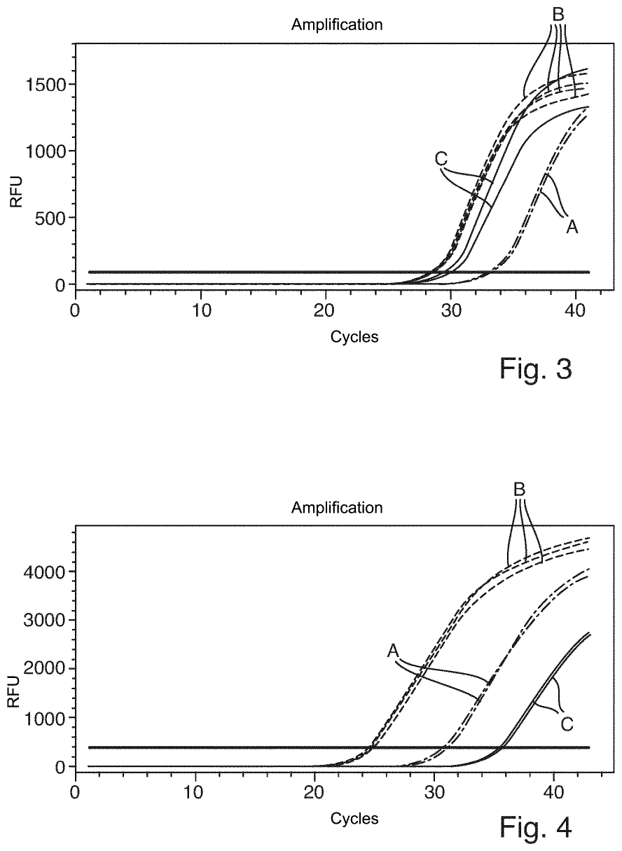

FIG. 3 shows amplification curves of different samples from Salmonella-containing water without concentration, after concentration by means of a filtration method and after concentration by means of the method according to the invention;

FIG. 4 shows amplification curves of different samples from MS2-phage RNA-containing water without concentration, after concentration by means of a filtration method and after concentration by means of the method according to the invention;

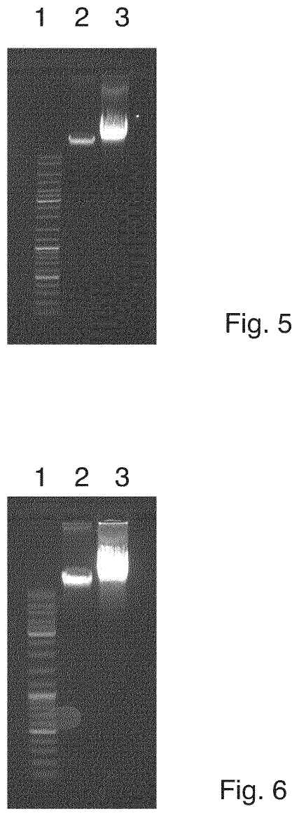

FIG. 5 shows a gel-electrophoretic representation of genomic DNA from a water sample without concentration and after concentration by means of the method according to the invention;

FIG. 6 shows a gel-electrophoretic representation of DNA eukaryotic cells from a water sample without concentration and after concentration by means of the method according to the invention; and

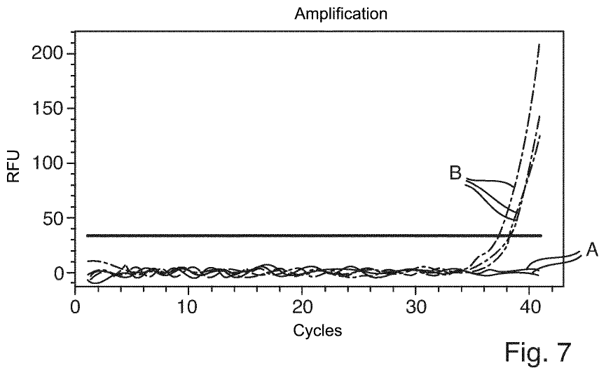

FIG. 7 shows amplification curves of different samples of water, which contains DNA in a very low concentration, without concentration and after concentration by means of the method according to the invention using superabsorber beads of different sizes.

The invention described here was based on the following unexpected observation. Commercially available so-called water pearls (commercially available inter alia under the names aqua beads, water beads or gel beads) were added to a volume of liquid of 1 liter. These water beads are formed from a superabsorber material. The liquid was surface water which had been removed from a firefighting water pond with suspended substances. After an incubation time, the beads had swollen to a multiple of their original volume. The volume of the liquid fraction of the mixture of the liquid and the water beads had reduced. Surprisingly, it was found that the suspended matter of the liquid was not absorbed by the swelling beads. The liquid fraction of the mixture, including the suspended matter (volume 400 ml), was transferred into a new vessel. A sample with a volume of 50 ml was removed from this liquid fraction.

A comparison sample with a volume of 50 ml was removed directly from the liquid, i.e., the mentioned surface water, without the liquid previously being concentrated according to the method described. Both samples were centrifuged at 5000×g for 10 min. The supernatant was removed and the sediment was used for a nucleic acid extraction. Nucleic acid extraction was effected by means of a commercial kit (innuprep Stool DNA Mini Kit; IST Innuscreen GmbH). The DNA of both samples was then examined by real-time PCR for the total bacterial germ count.

It was found here that fewer bacteria were detected in the untreated comparative sample than in the sample concentrated with the superabsorber. This means that the bacteria contained in the liquid were not absorbed into the water beads. They were concentrated and were a part of the remaining liquid fraction of the mixture of the liquid and the superabsorber beads. These observations could also be confirmed below for other biomolecules (viruses or eukaryotic cells): After addition of the superabsorber to a volume of liquid containing these biomolecules, the volume was concentrated and the biomolecules were concentrated in the remaining residual liquid. It was even more surprising that the method described can also concentrate proteins and free genomic DNA which are located in low concentrations in an aqueous solution.

The use of superabsorbers for concentrating a target substance, in particular biomolecules, in a polar liquid as solvent, for example water, for concentrating the target substance in the liquid is very simple and universally usable. A suitable method is described briefly with reference to FIG. 1 as follows:
1. Adding a superabsorber 2 to a volume of a liquid 1, in particular an aqueous liquid, or alternatively: adding the liquid to a superabsorber 2;
2. Incubating the mixture formed from the liquid 1 and the superabsorber 2 to concentrate (reducing the volume) the liquid fraction 3 of the mixture; and then
3. transferring at least some of the liquid fraction 3 of the mixture into a new vessel as a sample for further processing. The further processing can be, for example, a nucleic acid extraction, a measurement or also a direct analysis with a wide variety of technologies, such as NGS applications, immunological technologies, spectroscopic technologies, molecule spectroscopic or mass spectrometric technologies, etc.

Alternatively, the transferred portion of the liquid fraction can also be used as a product for further research purposes, for therapeutic purposes or as a crude product in a production process.

The degree of concentration and the speed of this process can be controlled very precisely by means of the type of superabsorber used, by means of the amount used, or by means of the incubation time and/or the incubation temperature.

With the method, the problem of processing low-concentrated samples can thus be easily solved for further processing of the target substances of interest, in particular biomolecules, or their detection. The method manages without expensive devices, for example ultracentrifuges, expensive ultrafiltration membranes, complex processes, such as PEG precipitation or generally precipitation reactions for concentration of nucleic acids, etc. In addition, the method can be used universally in relation to the target substances, in particular for biomolecules. A further advantage is that the superabsorbers are non-toxic and safe and are often also biodegradable. The method according to the invention makes it possible to greatly simplify the investigation of low-concentrated biomolecules.

However, further tests for concentrating genomic DNA contained in a sample liquid revealed a further effect. In these tests, an aqueous DNA solution having a volume of 500 μl was incubated with addition of a bead formed of a superabsorbent material (commercially available water pearls; diameter about 1 mm). With increasing incubation time, the volume of the sample is reduced and the concentration of the DNA is increased. The process was completed as the volume of the liquid was de facto zero. This result implies the idea of the loss of DNA. As a result, the observation was that, in a sample produced by adding 250 μl of water to the bead of superabsorber material and shaking the vessel briefly, DNA was measurable, namely at approximately twice the concentration in relation to the starting sample of 500 μl. It was thus shown that the DNA is not absorbed by the superabsorber material, but instead had been deposited on the outer surface of the bead formed from superabsorber material and can be recovered after addition of a liquid medium. It is thus possible not only to concentrate biomolecules in a sample liquid by means of the method according to the invention, but to adjust the biomolecules, in this case the DNA, in a targeted manner to a desired concentration by adding an aqueous solution, after complete liquid absorption by the absorber used.

On the basis of this observation, further possibilities for using superabsorbers for concentrating biomolecules are shown. In order to generate a sample of a sample liquid that can be used for a qualitative or quantitative analysis, which sample liquid contains biomolecules, such as eukaryotic cells, prokaryotic cells, viruses, phages or subcellular compartments, as the target substance to be determined qualitatively or quantitatively, the following method can be applied: Firstly, a first volume of the sample liquid can be treated with a superabsorber in such a way that the total liquid is absorbed by the superabsorber. The so-called water pearls already described are preferably used as superabsorbers. After incubation and complete liquid absorption, a lysis buffer is added to the superabsorber and the mixture thus produced is incubated. The type of lysis buffer and/or the incubation time can be selected as desired. Subsequently, the lysis buffer is separated from the superabsorber. The liquid sample thus obtained contains the target substance. The biomolecules contained as a target substance in the sample can be further lysed, as necessary. After lysis, the sample can be used for nucleic acid extraction. Under the precondition that the lysis has already been successfully implemented, the lysate is used for nucleic acid extraction without further incubation.

Some exemplary embodiments of the invention are described in more detail below.

### Exemplary Embodiment 1: Concentration of a Dirty Water Sample with a Volume of 1 Liter

The sample liquid used was dirty water from a firefighting water pond. Two volume units of 1 liter of the liquid each were transferred into a sample bottle.

For the concentration, 50 g of superabsorber beads obtainable commercially under the name “water beads” with a mass of about 0.006 g per bead and a diameter of about 1 mm were added into one of the sample bottles. After incubation, the remaining liquid fraction of about 650 ml was transferred into a new vessel. This was done by concentrating the volume of sample liquid originally used from 1000 ml to 650 ml. To verify that the concentration of bacteria could be increased in the concentrated sample liquid, two samples (samples 1 and 2) having a volume of 10 ml of the concentrated sample liquid and, for comparison, two samples of the non-concentrated sample liquid contained in the second sample bottle (samples 3 and 4) were centrifuged in a 15 ml reaction vessel at 5000 rpm for 10 min, and the supernatant was removed. The resulting sediment pellet was subsequently used for DNA extraction. DNA extraction was carried out using a commercial kit (innuPREP Stool DNA Kit; IST Innuscreen GmbH). The extracted DNA was used by real-time PCR to determine the total bacterial number. A commercial kit (innuDETECT Bacteria Quantification Assay; IST Innuscreen GmbH) was used as the detection system.

To determine the number of bacteria copies in the sample, the standard DNA was used from the assay.

FIG. 2 shows the amplification curves of the samples. The curves A (solid line) are the amplification curves of the three standards. The curves B (long and short lines) are the amplification curve of the samples 1 and 2 taken from the concentrated sample liquid, and the curves C (lines of equal length) are the amplification curve of the samples 3 and 4 taken from the untreated sample liquid. Table 1 specifies the Ct values and copy numbers for the individual standards and samples.

The results of real-time PCR demonstrate that, by the method according to the invention, an approximately 3-fold concentration of the total bacterial count in the sample is to be detected.

TABLE 1

| Sample | Ct value | Copy number |
|---|---|---|
| 1 (after concentration) | 21.2 | 4.3 × 10⁵ |
| 2 (after concentration) | 20.7 | 6.1 × 10⁵ |
| 3 (not concentrated) | 23.3 | 1.1 × 10⁵ |
| 4 (not concentrated) | 22.8 | 1.5 × 10⁵ |
| Standard 1 | 18.9 | 2 × 10⁶ |
| Standard 2 | 22.2 | 2 × 10⁵ |
| Standard 3 | 25.6 | 2 × 10⁴ |

### Exemplary Embodiment 2 Detection of Salmonella in Water Samples

Tap water with added Salmonella was used as the sample liquid, with 1×10⁶ Salmonella being added in each case as a spike to a volume of 10 ml of the tap water.

Two samples (sample 1 and 2) of 200 μl each were taken from such a non-concentrated sample liquid volume, in each case as comparative samples. Two further sample liquid volumes of 10 ml were concentrated by means of the method according to the invention. Superabsorber beads, obtainable commercially under the designation “water beads” and having a mass of about 0.006 g and a diameter of about 1 mm per bead, were used for the concentration. 40 pieces of the “water beads” were added to each of the two sample liquid volumes. By incubating the mixture thus obtained, the liquid fraction thereof was concentrated to a volume of about 500 μl. From this liquid fraction, 2× in each case 200 μl was used for the subsequent DNA extraction (samples 3 to 6).

In parallel thereto, a further sample liquid volume of 10 ml was processed by means of a standard method using a filtration membrane. For this purpose, the sample liquid volume was filtered via a filter membrane (0.45 μm MCE membrane; Millipore) using a vacuum pump. The filter was cut and admixed with 600 μl of 1×PBS solution and homogenized in a lysis tube using a homogenizer (SpeedMill, Analytik Jena GmbH). Two samples, each having a volume of 200 μl taken from the approximately 500 μl of liquid obtained after homogenization, were likewise used for DNA extraction (samples 7 and 8).

DNA extraction was carried out by means of an automated method on the KingFisher Flex (Thermo Fisher) machine and a commercially available kit (deltaPREP AniPath DNA/RNA Kit KFFLX; IST Innuscreen GmbH). The extracted DNA was used by real-time PCR to detect the Salmonella. A commercial kit (innuDETECT Salmonella enterica Assay; IST Innuscreen GmbH) was used as the detection system.

FIG. 3 shows the amplification curves of the samples. The curves A (long and short lines) show the curves of the amplification curves of the samples 1 and 2 of the non-concentrated sample liquid. The curves B (lines of equal length) are the amplification curves of samples 3 to 6 of the sample liquid concentrated by the method according to the invention. The curves C (solid line) are the amplification curves of samples 7 and 8 obtained by filter-based enrichment.

Table 2 specifies the Ct values for the individual samples.

TABLE 2

| Sample | Ct value |
|---|---|
| 1 (without concentration) | 33.3 |
| 2 (without concentration) | 33.1 |
| 3 (after concentration) | 28.4 |
| 4 (after concentration) | 28.3 |
| 5 (after concentration) | 28.2 |
| 6 (after concentration) | 28.2 |
| 7 (filter-based) | 29.9 |
| 8 (filter-based) | 29.4 |

The differences between the Ct-values show mathematically a 32-fold concentration of the starting sample with regard to the Salmonella number. The enrichment of the bacteria on a filter shows a poorer efficiency compared to the method according to the invention.

### Exemplary Embodiment 3: Detection of MS2 Phage RNA in Water Samples

Tap water with added MS2 bacteriophages was used as the sample liquid, wherein 5 μl of an MS2 bacteriophage solution (Leibniz Institute DSMZ: DSM 13767) was added as a spike in each case to a volume of 10 ml of the tap water.

Two samples (samples 1 and 2) of 200 μl each were taken from such a sample liquid volume as comparative samples. Two further sample liquid volumes of 10 ml each of the sample liquid were concentrated by means of the method according to the invention. Superabsorber beads, obtainable commercially under the designation “water beads” and having a mass of about 0.006 g and a diameter of about 1 mm per bead, were used for the concentration. 40 pieces of the “water beads” were added to each of the two sample liquid volumes. By incubating the mixture thus obtained, the liquid fraction thereof was concentrated to a volume of about 500 μl. From this liquid fraction, 2× in each case 200 μl was used for the subsequent phage RNA extraction (samples 3 to 6).

In parallel thereto, a further sample liquid volume of 10 ml was processed by means of a standard method using a filtration membrane. For this purpose, the sample liquid volume was filtered via a filter membrane (0.45 μm MCE membrane; Millipore) using a vacuum pump. The filter was cut and admixed with 600 μl of 1×PBS solution and homogenized in a lysis tube using a homogenizer (SpeedMill, Analytik Jena GmbH). Two samples, each having a volume of 200 μl taken from the approximately 500 μl of liquid obtained after homogenization, were likewise used for phage RNA extraction (samples 7 and 8).

The phage RNA extraction was carried out by means of an automated method on the KingFisher Flex (Thermo Fisher) machine and a commercially available kit (deltaprep AniPath DNA/RNA Kit KFFLX; IST Innuscreen GmbH). The extracted phage RNA was used by real-time PCR to detect the MS2 phage RNA. A commercial kit (innuDETECT Internal Control DNA/RNA Assay; IST Innuscreen GmbH) was used as the detection system. For the reverse transcription and amplification of the MS2 phage RNA, a commercial OneStep-RT-MasterMix was used (innuDRY qRT-PCR MasterMix Probe; IST Innuscreen GmbH).

FIG. 4 shows the amplification curves of the samples. The curves A (long and short lines) show the profiles of the amplification curves of the samples 1 and 2 of the non-concentrated sample liquid. The curves B (lines of equal length) are the amplification curves of samples 3 to 6 of the sample liquid concentrated by the method according to the invention. The curves C (solid line) are the amplification curves of samples 7 and 8 obtained by filter-based enrichment.

Table 3 indicates the Ct values for the individual samples:

TABLE 3

| Sample | Ct value |
|---|---|
| 1 (without concentration) | 30.8 |
| 2 (without concentration) | 31.3 |
| 3 (after concentration) | 24.6 |
| 4 (after concentration) | 25.1 |
| 5 (after concentration) | 24.6 |
| 6 (after concentration) | 24.5 |
| 7 (filter-based) | 35.7 |
| 8 (filter-based) | 35.4 |

The differences between the Ct-values show mathematically a 50-fold concentration of the starting sample. The filter-based enrichment appears to be suitable for the enrichment of the phages only very poorly.

### Exemplary Embodiment 4: Concentration of Genomic DNA in a Water Sample and Spectrophotometric Measurement

To verify that the method according to the invention is also suitable for concentration of genomic DNA in a sample liquid, genomic DNA was isolated from a blood sample. The DNA was dissolved in water and the concentration of the DNA was set to 10 ng/μl. The starting volume of the sample liquid thus prepared was 2 ml. The concentration was carried out by adding 10 pieces of commercially available “water beads” (mass per piece about 0.006 g; diameter: about 1 mm). After a short incubation time, the volume of the sample liquid was concentrated to a residual volume of 500 μl and a first spectrophotometric measurement for determining the concentration of the DNA was carried out in the liquid fraction of the mixture. After the incubation time had been extended, the liquid fraction of the mixture was concentrated further up to 250 μl and a second spectrophotometric measurement for determining the concentration of the DNA was carried out in the liquid fraction of the mixture. The results are summarized in Table 4.

TABLE 4

| Sample | DNA concentration |
|---|---|
| Sample liquid before concentration | 10 ng/μl |
| Sample liquid concentrated to 500 μl | 37 ng/μl |
| Sample liquid concentrated on 250 μl | 70 ng/μl |

The data show that, after concentration, the measured values correspond to the theoretical values with only slight deviation.

### Exemplary Embodiment 5: Concentration of Genomic DNA from a 2 ml Sample and Detection of the Increase in Concentration on an Agarose Gel

To verify that the method according to the invention is also suitable for concentration of genomic DNA of a sample liquid, genomic DNA was isolated from a blood sample. The DNA was dissolved in water and the concentration of the DNA was set to 10 ng/μl. The starting volume of the sample liquid thus prepared was 2 ml. A first sample was taken from this starting volume as a comparison sample for gel electrophoresis. The concentration of the starting volume was carried out by adding 10 pieces of commercially available “water beads” (mass per piece about 0.006 g; diameter: about 1 mm). After a short incubation time, the liquid fraction of the mixture thus produced was concentrated to a residual volume of 250 μl. A second sample removed from this residual volume was subsequently shown in comparison to the starting sample on an agarose gel.

FIG. 5 shows the gel-electrophoretic representation of the DNA, wherein in the first trail (1) the DNA conductor, in the second trail (2) the comparison sample, and in the third trail (3) the second sample from the concentrated residual volume are located. The gel pattern clearly shows the strongly increased amount of DNA after the concentration compared to the non-concentrated sample liquid.

### Exemplary Embodiment 6: Enrichment of Eukaryotic Cells in a Sample Liquid and Subsequent Extraction of the DNA from these Cells

To verify that the method according to the invention is also suitable for concentration of eukaryotic cells present in a sample liquid, nucleated cells were isolated from a blood sample. The cells were then resuspended completely in an initial volume of 2 ml of water. Before concentration of the sample liquid, 200 μl of the starting cell suspension were removed as a comparison sample and used for DNA extraction.

The sample liquid was concentrated by adding 8 superabsorber beads commercially available under the name “water beads” and having a mass of about 0.009 g and a diameter of about 2 mm. After a short incubation time, the starting volume of the sample liquid was concentrated to a residual volume of about 400 μl. Of this approximately 400 μl, 200μ were removed as a sample and used for DNA extraction. DNA extraction was carried out by means of a commercial kit (innuPREP DNA Mini Kit 2.0; IST Innuscreen GmbH). The DNA was measured spectrophotometrically and analyzed on an agarose gel.

The results of spectrophotometric measurements and the determined DNA amount in the corresponding samples are summarized in Table 5.

TABLE 5

| | DNA | Ratio | Ratio |
|---|---|---|---|
| Sample | amount | A₂₆₀:A₂₈₀ | A₂₆₀:A₂₃₀ |
| Extraction of 200 μl of | 2.1 μg | 1.8 | 2.2 |
| the starting volume | | | |
| (comparative sample) | | | |
| Extraction of 200 μl of the | 9.9 μg | 1.9 | 2.3 |
| residual volume concentrated | | | |
| to about 400 μl | | | |

FIG. 6 shows the gel-electrophoretic representation of the DNA, wherein in the first trail (1) the DNA conductor, in the second trail (2) the comparison sample, and in the third trail (3) the second sample from the concentrated residual volume are located.

The data show that eukaryotic cells can be enriched from a sample by means of the methods according to the invention. The DNA is of high quality.

### Exemplary Embodiment 7: Concentration of a Protein Solution

To verify that the method according to the invention is also suitable for the concentration of proteins in a sample liquid, an aqueous albumin solution was prepared. The protein concentration was determined spectrophotometrically at 280 nm to be 11.8 mg/ml in the untreated starting solution. Different concentrations were carried out. For this purpose, an initial volume of 100 μl of the protein solution was transferred in each case into a reaction vessel, and the solution was incubated in each case with a bead of commercially available “water beads” (mass about 0.006 g; diameter: about 1 mm) over different periods of time. This led to the concentration of the starting solution from 100 μl to about 60 μl, about 40 μl and about 20 μl. The residual volumes thus concentrated were measured as individual samples subsequently at 280 nm, and the protein concentration was determined.

The results are summarized in Table 6.

TABLE 6

| | Protein concentration |
|---|---|
| | determined from |
| | photometric measurement |
| Sample | at 280 nm in mg/ml |
| Starting solution | 11.8 |
| in a sample concentrated to about 60 μl | 18.2 |
| in a sample concentrated to about 40 μl | 24.1 |
| in a sample concentrated to about 20 μl | 46.3 |

It has been found that, by means of the method, proteins can also be concentrated and the concentrations increase continuously depending on the concentration.

### Exemplary Embodiment 8: Increase in Sensitivity of Real-Time PCR after Concentration of a Sample with Human DNA of Very Low Concentration

To verify that the method according to the invention is also suitable for the concentration of solutions with very low concentrations of DNA, a sample liquid was prepared with human genomic DNA in a very low concentration. The DNA concentration of the sample was 8 ng/μl, which corresponds to about the amount of genomic DNA of a diploid eukaryotic cell. For the concentration, 100 μl of the sample liquid were used as initial volumes. 1 piece of commercially available “water beads” (mass about 0.006 g; diameter: about 1 mm) was added to a first starting volume of the sample liquid and 1 piece of commercially available “water beads” (mass about 0.009 g; diameter: about 2 mm) was added to a second starting volume of the sample liquid. After a short incubation, the liquid fractions of the two mixtures were concentrated to about 10 μl. A comparison sample (sample 1) taken from the non-concentrated sample liquid and samples removed from the concentrated liquid fractions of the two mixtures (samples 2-5) were used in real-time PCR for detection of a human-specific target sequence (single copy gene estrogen receptor 1, in-house method).

The amplification curves of the individual samples are shown in FIG. 7. The curves A (solid line) in color are the amplification curves of the comparison sample (duplicate determination); the curves B (long and short lines) are the amplification curves of samples 2 to 5.

Table 7 specifies the Ct values for the individual samples.

TABLE 7

| Sample | Ct value |
|---|---|
| 1 (before concentration) | No Ct |
| 1 (before concentration) | No Ct |
| 2 (after concentration Ø 1 mm “water beads”) | 38.2 |
| 3 (after concentration Ø 1 mm “water beads”) | 37.4 |
| 4 (after concentration Ø 2 mm “water beads”) | 38.4 |
| 5 (after concentration Ø 2 mm “water beads”) | 38.5 |

Sampling from the starting solution did not achieve an amplification result, since the concentration of the DNA was too low. The DNA from the concentrated samples was able to detect the single copy gene, which shows the success of the method according to the invention.

### Exemplary Embodiment 9: Production of a Sample by Incubating a DNA Solution with a Superabsorber Until Complete Liquid Absorption and Subsequent Adjustment of the Sample Concentration by Addition of an Aqueous Solution

A DNA solution with a concentration of 100 ng/μl was prepared as a sample liquid (lambda DNA). Two commercially available “water beads” (0.006 g; diameter about 1 mm) were added to 500 μl of this DNA solution, and the first mixture thus obtained was incubated until the liquid fraction of the mixture had completely disappeared. Thereafter, 250 μl of a buffer (10 mM Tris HCl, pH 8.5) were added to the remaining “water beads”. The second mixture produced in this way was mixed by means of a vortex mixer and then the liquid fraction of the second mixture was separated from the “water beads” as a sample to be analyzed, and was analyzed spectrophotometrically.

Table 8 indicates the original DNA concentration in the original DNA solution serving as sample liquid and the spectrophotometrically determined concentration in the sample.

TABLE 8

| Sample | DNA concentration |
|---|---|
| Sample liquid 500 μl | 100 ng/μl |
| Sample after complete fluid reduction and | 181 ng/μl |
| subsequent addition of 250 μl Tris buffer | |

The data show that the concentration determined in the sample after concentration and re-suspension corresponds to the theoretical values with only a small deviation.

### Exemplary Embodiment 10: Concentration of a Sample Liquid for Detecting a Protein (CRP) by Means of an Immunological Detection Method

A dilution of the C-reactive protein (CRP) in a PBS buffer solution was used as sample liquid. The immunological detection of the C-reactive protein in various samples produced from the sample liquid was effected by means of a CRP ELISA kit from Bio-Techne GmbH (h C-Reactive Protein DuoSet, DY1707). The starting sample served as the positive standard control (human CRP) of the kit. The measurements were carried out in an ELISA reader (Thermofisher) at 405 nm. Three solutions (samples 1, 2, and 3) of different concentrations of CRP were produced from the starting sample. A first fraction of about 80 μl was removed from each of these samples, diluted with 120 μl of the dilution buffer of the kit and placed on the ELISA plate. A further fraction of 500 μl in each case was removed from the samples 1, 2 and 3, and in each case 6 “water beads” (0.006 g, diameter 1 mm) were added and incubated until the samples 1A, 2A and 3A thus obtained were concentrated to a volume of about 70-80 μl. These concentrated samples 1A, 2A and 3A were likewise diluted with 120 μl of the dilution buffer of the kit and placed on the ELISA plate. The solutions of samples 1, 2, 3, 1A, 2A, and 3A applied to the ELISA plate were measured semi-quantitatively in addition to a standard series [S1-S4, per 200 μl] according to the instructions of the manufacturer. The results are summarized in Table 9.

TABLE 9

| | CRP concentration | CRP amount (pg) | | |
|---|---|---|---|---|
| | (pg/ml) (theoretically | (theoretically | | CRP concentration |
| Sample | calculated in | calculated in | Measurement at | calculated from |
| no./St no. | samples 1A-3A) | samples 1A-3A) | 405 nm | measurement |
| 1 | 12000 | 960 | 0.710 | 3700 |
| 2 | 1200 | 96 | 0.185 | 367 |
| 3 | 120 | 9.6 | 0.079 | n.D |
| 1A | 75000 | 6240 | 1.006 | 7800 |
| 2A | 7500 | 624 | 0.482 | 2000 |
| 3A | 750 | 62.4 | 0.088 | 120 |
| S1 | 2000 | 40 | 0.484 | 2000 |
| S2 | 1000 | 20 | 0.322 | 1000 |
| S3 | 500 | 10 | 0.215 | 500 |
| S4 | 250 | 5 | 0.152 | 250 |

Due to the inadequate binding capacity, span and linearity of the ELISA method, the theoretically expected protein amounts could not be measured exactly. The theoretically calculated enrichment was the factor 6.5. The enrichment rate measured by ELISA is between 2.1 and 5.4.

The concentration of the non-measurable sample 3 was able to be determined in the sample 3A produced from sample 3 by enrichment. It is thus found that it is possible by means of the method according to the invention to concentrate a specific target protein in a sample liquid and to detect it by ELISA. A sensitivity advantage can thus be achieved via the concentration.

1-22. (canceled)

23. A method for producing a sample containing at least one target substance from a first volume of liquid of a sample liquid containing the at least one target substance by concentration of the target substance in the first volume of liquid, the method comprising:
adding a superabsorber to the first volume of liquid or adding the first volume of liquid to the superabsorber;
incubating a first mixture formed from the superabsorber and the first volume of liquid; and
removing a sample of the liquid fraction of the first mixture present after incubation.

24. The method of claim 23, further comprising:
after the incubating of the first mixture, adding a second volume of liquid of an aqueous solution to the remaining superabsorber or adding the remaining superabsorber to the second volume of liquid and producing a second mixture of the superabsorber and the second volume of liquid; and
removing a sample of a liquid fraction of the second mixture.

25. The method according to claim 24, further comprising:
after the adding of the second volume of the liquid, incubating the second volume of liquid before the removing of the sample of the liquid fraction of the second mixture.

26. The method according to claim 24,
wherein the aqueous solution includes a lysis buffer.

27. The method according to claim 24,
wherein the incubation of the first mixture is carried out until the liquid fraction of the first mixture has completely disappeared.

28. The method according to claim 23,
wherein the first volume of liquid contains a polar liquid.

29. The method according to claim 28,
wherein the polar liquid is water.

30. The method according to claim 23,
wherein the target substance is a biomolecule.

31. The method according to claim 23,
wherein the target substance is selected from the group formed of: eukaryotic cells, components of eukaryotic cells, prokaryotic cells, components of prokaryotic cells, subcellular vesicles, bacteriophages, viruses, or virus components, toxins, antibodies, nucleic acids, and proteins.

32. The method according to claim 23,
wherein the superabsorber includes a plastic that absorbs water to form a hydrogel.

33. The method according to claim 32,
wherein the plastic does not take up any biomolecules, in particular substantially no eukaryotic cells, components of eukaryotic cells, prokaryotic cells, components of prokaryotic cells, subcellular vesicles, bacteriophages, viruses, or virus components, toxins, antibodies, nucleic acids, or proteins.

34. The method according the claim 33,
wherein the plastic does not take up any eukaryotic cells, components of eukaryotic cells, prokaryotic cells, components of prokaryotic cells, subcellular vesicles, bacteriophages, viruses, or virus components, toxins, antibodies, nucleic acids, or proteins.

35. The method according to claim 23,
wherein the superabsorber is used in the form of particles or in the form of geometric bodies.

36. The method according to claim 23,
wherein the superabsorber is used in the form of water pearls, hydrobeads, aquapearls, aquabeads, water beads, or gel beads.

37. The method according to claim 35,
wherein the particles have a diameter between 100 micrometers (μm) to 5000 μm.

38. The method according to claim 24,
wherein the volume of the liquid fraction of the first or second mixture remaining after incubation is controlled by the duration of incubation and/or by the type and/or amount of the superabsorber and/or by the temperature of the mixture prevailing during incubation.

39. The method according to claim 23,
wherein the superabsorber is used in the form of particles, and wherein the volume of the liquid fraction remaining after incubation is controlled by the size and/or number of particles.

40. The method according to claim 23,
wherein the sample of the liquid fraction is used as a crude product or product in an experimental method or in a production process.

41. A kit, comprising:
at least one container; and
a superabsorber into which an initial volume of a liquid can be added for concentration.

42. A method for the qualitative or quantitative determination of at least one target substance in a sample liquid, the method comprising:
producing a sample containing the at least one target substance from a first volume of liquid of a sample liquid containing the at least one target substance by:
adding a superabsorber to the first volume of liquid or adding the first volume of liquid to the superabsorber;
incubating a first mixture formed from the superabsorber and the first volume of liquid; and
removing a sample of the liquid fraction of the first mixture present after incubation; and
qualitatively or quantitatively detecting the at least one target substance on the basis of the sample by means of at least one of the following methods: nucleic-acid-based detection methods, in particular PCR-based, real-time PCR-based or digital PCR-based methods, sequencing, immunological detection methods, in particular ELISA, lateral flow tests, microbiological analyses, microscopic methods, mass spectrometry, detection methods by means of optical, spectroscopic, or electrochemical sensors, and flow cytometry.
